(19) United States
(12) Patent Application Publication
(10) Pub. No.: US 2025/0339317 A1
(43) Pub. Date: Nov. 6, 2025

(54) LINEAR PATH IMAGE PROJECTION ONTO RETINA FROM ELECTRONIC INTRAOCULAR LENS (IOL)

(71) Applicant: Glaive Medical Optics Inc., Carlisle, MA (US)

(72) Inventors: Wayne H. WOODS, JR., Carlisle, MA (US); Christopher SHELBY, Shreveport, LA (US); Alexander J. MELVILLE, Chelmsford, MA (US)

(21) Appl. No.: 19/195,967

(22) Filed: May 1, 2025

Related U.S. Application Data
(60) Provisional application No. 63/640,982, filed on May 1, 2024.

(51) Int. Cl. A61F 9/08; A61F 2/48; G09G 3/00; G16H 20/40; G16H 40/67; G16H 50/50

(52) U.S. Cl. CPC A61F 9/08; A61F 2/482; G09G 3/002; G16H 20/40; G16H 40/67; G16H 50/50; A61F 2250/0002; G09G 2380/08

(57) ABSTRACT

An electronic intraocular lens configured to be implanted in an eye includes: an imaging system that receives visible light incoming to the eye; and a projection system including a display and a lens that are configured to generate and project an image onto a retina of the eye in which the device is implanted, the image being based on the light received by the imaging system.

### RELATED APPLICATIONS

This application claims domestic priority to U.S. provisional application No. 63/640,982, filed May 1, 2024. This application incorporates by reference the entire contents of U.S. application Ser. No. 18/384,585, filed Oct. 27, 2023, published as US20240138673A1. This application incorporates by reference the entire contents of U.S. application Ser. No. 18/373,669, filed Sep. 27, 2023, published as US20250099299A1.

### BACKGROUND

The present invention relates generally to ocular implants and, more particularly, to an ocular prosthetic comprising a surgically implanted ocular optical array that can be used in both therapeutic and diagnostic applications.

Being able to target/stimulate specific areas of the retina surface is desirable and difficult to achieve. Approaches to doing this have included chips that directly interface with the neurons in the retina surface. In this disclosure, devices and methods are described that are much less surgically invasive compared to such alternatives.

### SUMMARY

In an aspect of the invention, there is a device configured to be implanted in an eye, the device comprising: an imaging system that receives visible light incoming to the eye; and a projection system that is configured to generate and project an image onto a retina of the eye in which the device is implanted, the image being based on the light received by the imaging system. The imaging system may comprise a CCD/imaging chip or similar imaging device.

In an embodiment, the device further comprises control circuitry that causes the projection system to project the image onto a determined area of the retina.

In an embodiment, the projection system comprises a display, such as an LED or LCD panel, or similar, that comprises a plurality of individually controllable light emitting elements.

In an embodiment, the projection system comprises a lens arranged in a Z-direction over the display, i.e., between the display and the retina of an eye when the device is implanted in the eye.

In an embodiment, the determined area of the retina is a healthy area of the retina.

In an embodiment, the control circuitry determines the determined area of the retina using a stored mapping.

In an embodiment, the imaging system, the control circuitry, and the projection system are arranged in a chip stack.

In an embodiment, the imaging system is at a first side of the chip stack, and the projection system is at a second side of the chip stack opposite the first side of the chip stack.

In an embodiment, the device comprises a body comprising a central portion and tabs extending outward from the central portion, and the chip stack is in the central portion.

In an embodiment, the device further comprises a wireless communication antenna that is configured to receive wireless communication signals from outside the device.

In an embodiment, the control circuitry is configured to program the mapping based on the wireless communication signals.

In an embodiment, the device further comprises a rechargeable battery that is configured to power the imaging system, the control circuitry, and the projection system.

In an embodiment, the rechargeable battery is configured to be recharged wirelessly from a charging system located outside the eye.

In an embodiment, the device is configured to be implanted in a capsular bag of the eye.

In an embodiment, the device is configured to be implanted in a ciliary sulcus of the eye.

In an embodiment, the device is configured to be implanted in an anterior chamber of the eye anterior to the iris.

In an embodiment, a method comprises implanting the device into the eye.

In an embodiment, a method of using the device comprises: causing the device to project a diagnostic image on different locations of the retina of the eye; receiving patient feedback for each of the different locations; creating a mapping of the retina of the eye based on the feedback; and programming the mapping into the device.

In an embodiment, the method of using the device comprises optimizing the mapping using artificial intelligence.

In an embodiment of the method of using the device, the mapping maps the retina into functional areas and non-functional areas.

In an embodiment of the method of using the device, the device is configured to control one or more elements of the projection system based on the mapping to project an image onto a functional area of the retina to reduce or eliminate a scotoma caused by a non-functional area of the retina.

In an embodiment, a device according to any of the aspects above comprises a body made of acrylic and/or silicone lens material.

In an embodiment, a device according to any of the aspects above comprises a single piece lens.

In an embodiment, a device according to any of the aspects above comprises a body having dimensions of 1 mm≤TH≤3 mm and 1 mm≤W≤10 mm. In one example, a device according to any of the aspects above comprises a body having dimensions of 1 mm≤TH≤5 mm and 1 mm≤W≤10 mm. In another example, a device according to any of the aspects above comprises a body having dimensions of 1 mm≤TH≤10 mm and 1 mm≤W≤10 mm.

In an embodiment, a device according to any of the aspects above comprises an imaging chip comprising the imaging system, a control chip comprising the control circuitry, a chip comprising the projection system, wherein the chips are arranged in a chip stack. The chips may be made using semiconductor fabrication materials and techniques, including but not limited to Si, InP, GaAs, Liquid Crystal materials, and BGA/C4/micro-BGA, through substrate (or silicon) vias (TSVs), micro-TSVs, and solder or oxide bonding techniques.

In an embodiment, a device according to any of the aspects above comprises a wireless communication antenna (e.g., for receiving programming signals) and/or an inductive coupling coil (e.g., for wireless charging) embedded in the material of the body.

### BRIEF DESCRIPTION OF THE DRAWINGS

Aspects of the present invention are described in the detailed description which follows, in reference to the noted plurality of drawings by way of non-limiting examples of exemplary embodiments of the present invention.

FIG. 1 illustrates a projection system in accordance with aspects of the present invention.

FIG. 2 shows an example of an IOL that includes a projection system in accordance with aspects of the present invention.

### DETAILED DESCRIPTION

The particulars shown herein are by way of example and for purposes of illustrative discussion of the embodiments of the present invention only and are presented in the cause of providing what is believed to be the most useful and readily understood description of the principles and conceptual aspects of the present invention. In this regard, no attempt is made to show structural details of the present invention in more detail than is necessary for the fundamental understanding of the present invention, the description taken with the drawings making apparent to those skilled in the art how the several forms of the present invention may be embodied in practice.

The present invention relates generally to ocular implants and, more particularly, to surgically implanted electronic intraocular lens (IOL) that can be used in both therapeutic and diagnostic applications. In embodiments, a device comprises a projection system and control electronics that are configured to selectively aim projection of images onto one or more desired locations on a retina of an eye in which the device is implanted. In embodiments, an imaging system is integrated in a single assembly with the projection system and control electronics. In embodiments, the imaging system is configured to receive light coming into the eye, and the control electronics and projection system are configured to project an image onto the retina wherein the projected image corresponds to the light received by the imaging system. In embodiments, the imaging system receives an image of what the user's eye would normally see (i.e., normally meaning a healthy eye), and the control electronics and projection system are configured to project this same image (or a portion of the image, or a digitally altered/enhanced/manipulated portion of the image) onto the retina at a desired/determined good location of the retina. In this manner, the implanted device may serve to redirect the incoming image away from a bad location of the retina to a good location on the retina. As used herein, a bad location of the retina refers to damaged portion of the retina that can no longer sec (e.g., can no longer absorb light to a degree sufficient to provide sight to the person). In this way, when the device is implanted in an eye of a patient, the patient has vision which tracks with eyeball direction as opposed to, for example, an imaging system mounted on a pair of glasses and communicated to the microlens array from a wired/tethered or wireless network bridge.

Focusing light on the retina from a thin intraocular lens (IOL) is challenging while trying to maintain a suitably small thickness of the IOL. Light focusing elements such as lenses, etc. generally require a substantial distance to operate effectively. For example, a lens may need to be placed several focal lengths away from the image or target retina. That can be impractical for a rollable/collapsible IOL that rolls-up or folds-up to fit within a 2-3 mm incision when being implanted in the eye.

Images must be able to be shifted to different parts of the retina as the clinical placement of the image source in the electronic IOL may not exactly match where the image needs to be. Ideally, the image system needs to be compact and also allow image placement while also maximizing the number of pixels utilized in such a display.

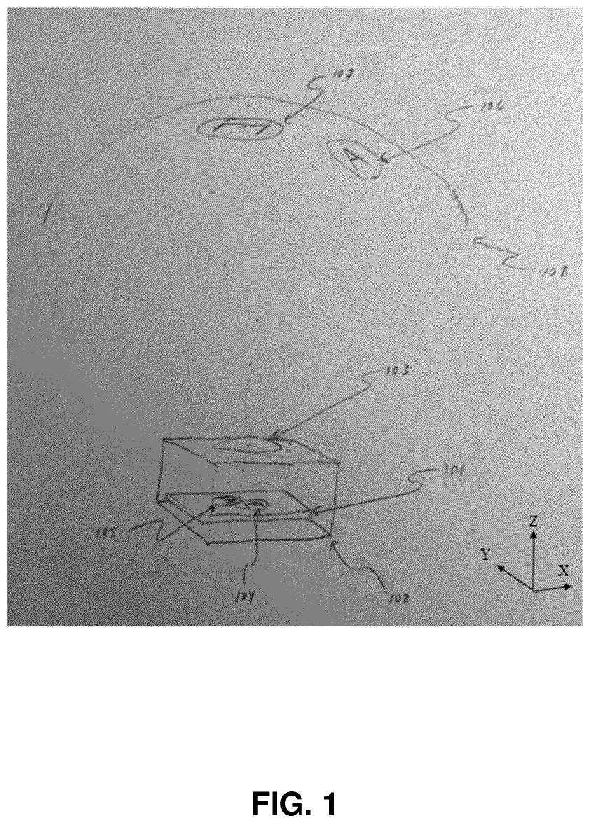

FIG. 1 illustrates a projection system in accordance with aspects of the invention relative to a projection surface 108 that corresponds to a retina of an eye in which a device including the projection system is implanted. The projection system may be used for projection of sub-image projection onto the retina 108.

With continued reference to FIG. 1, in embodiments the projection system includes an enclosure 102, a lens 103, and a display 101. In embodiments, only a subset of the display 101 needs to be turned on at a given time. This lowers power consumption and also allows the image to be placed preferentially on the retina. In example shown in FIG. 1, the display 101 activates elements (e.g., pixels) that display a first image 104 comprising the letter “E” in a circle. In this example, the light of the first image 104 emanates from the display 101 and through the lens 103, which causes the light to be projected onto the retina at a first location 107. In another example shown in FIG. 1, the display 101 activates elements (e.g., pixels) that display a second image 105 comprising the letter “A” in a circle. In this example, the light of the second image 105 emanates from the display 101 and through the lens 103, which causes the light to be projected onto the retina at a second location 106. The display 101 can be controlled to display the images 104 and 105 at different times or at the same time. In these examples, and as shown in FIG. 1, because of the different positions of first image 104 and second image 105 on the display 101 relative to the lens 103, the corresponding projected images are at different locations 106 and 107 on the retina 108. In embodiments, by shifting (e.g., selectively controlling) where a source image is created on the display 101, different regions of the retina 108 can be reached by the projection via the lens 103. This lowers power consumption and also allows the image to be placed preferentially on the retina 108, e.g., at determined good locations of the retina.

In embodiments, the enclosure 102 comprises a box or similar enclosure that supports the lens 103 above the display 101 so that the lens 103 is between the display 101 and the retina 108 when an IOL including the projection system is implanted in an eye. The enclosure 102 may comprise one or more walls and a “lid” that supports the lens 103. The one or more walls and the lid may be opaque material. The projection system including the display 101, enclosure 102, and lens 103 may be referred to as a linear path projection system because there is a linear optical path within the enclosure 102 from the display 101 to the lens 103 (i.e., there is an optical path that begins at the display 101 and that ends at the lens 103 and that extends continuously in a straight line for the entire distance between the display 101 and the lens 103). The interior of the enclosure 102 between the display 101 and the lens 103 is transparent and may comprise an optically transparent solid material, air, or inert gas, for example. In embodiments, the lens 103 comprises a double-convex lens or a plano-convex lens, although implementations are not limited to these examples.

In accordance with aspects of the invention, individual pixels of the display 101 can be selectively turned on (e.g., emitting light) or off (not emitting light) at a given time. As such, a first subset of pixels of the display 101 can be turned on concurrently with a second subset of the pixels of the display 101 being turned off. Due to the different positions of the activated pixels of the display 101 relative to the lens 103, combined with the optical characteristics of the lens 103 (e.g., index of refraction, focal length, etc.), a direction of light transmitted through the lens 103 can be varied based on which ones of the pixels of the display 101 are included in the first subset (i.e., turned on) and which ones of the pixels of the display 101 are included in the second subset (i.e., turned off) at any given time. In this manner, the display 101 and lens 103 can be used to project light in a particular direction outward from the lens 103.

Using one of the examples from FIG. 1, a first subset of the pixels that are turned on may comprise pixels that generate the first image 104 on the display 101, and a second subset of pixels that are turned off may comprise all other pixels of the display 101. In this example, the projection system including the display 101 and the lens 103 projects the corresponding image onto the retina at location 107.

Using another one of the examples from FIG. 1, a first subset of the pixels that are turned on may comprise pixels that generate the second image 105 on the display 101, and a second subset of pixels that are turned off may comprise all other pixels of the display 101. In this example, the projection system including the display 101 and the lens 103 projects the corresponding image onto the retina at location 106.

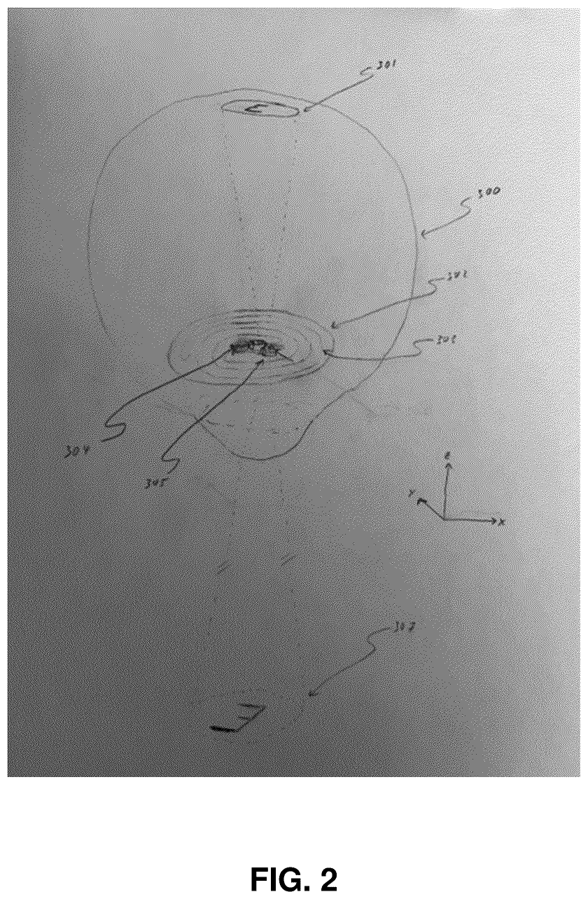

FIG. 2 shows an example of an IOL 302 that includes a projection system 304, such as that shown in FIG. 1, in accordance with aspects of the present invention, the IOL 302 being implanted in an eye 300. The projection system 304 of the electronic IOL 302 projects an image 301 onto the retina of the eye 300. The source image 307 that the eye is looking at is received by the imaging system 305 on the opposite side of the electronic IOL 302 from the projection system 304. The projection system 304 reproduces the electronic image received from the imaging system 305 and projects it onto the back of the eye 300. Examples of wireless charging coils/wires embedded into the flexible IOL 302 are also shown at element 302. The projection system 304 may be aligned with the imaging system 305 in the Z-direction although this is not required. In some implementations the projection system 304 is not aligned with the imaging system 305 in the Z-direction and instead is offset in the X-direction and/or the Y-direction.

In this manner, implementations of the present invention may be used to provide a structure for allowing image steering for an electronic IOL to the retina. This advantageously allows a thinner lenses/optical system to focus the light on the retina. Implementations may also be used to provide a structure, micro-lenses, sub-image of display, IOL, and micro-LED system, included a small, pixelated version of the device described herein.

Implementations of the projection system in accordance with FIG. 1 may be used in the devices 500/600/700/1600 described in U.S. application Ser. No. 18/384,585 published as US20240138673A1. For example, implementations of the projection system in accordance with FIG. 1 may be used in the device 1600 of FIG. 16 of in U.S. application Ser. No. 18/384,585 published as US20240138673A1, replacing elements 1645 and 1650, with control circuitry 1640 configured in a manner to control the display 101 of FIG. 1 in the manner described herein. By using a single display 101 and a single lens 103 instead of an array comprising multiple lenses, embodiments of the present invention are simpler (e.g., less complex) to implement compared to an IOL device including a microlens array comprising multiple lenses.

Implementations of an IOL in accordance with FIG. 2, including the projection system in accordance with FIG. 1, may be used with the methods of implant described at FIGS. 5-7 of U.S. application Ser. No. 18/384,585 published as US20240138673A1. Implementations of an IOL in accordance with FIG. 2, including the projection system in accordance with FIG. 1, may be used with the methods of retina mapping described at FIGS. 8-13 and 17A-C of U.S. application Ser. No. 18/384,585 published as US20240138673A1. Implementations of an IOL in accordance with FIG. 2, including the projection system in accordance with FIG. 1, may be used with the methods of charging and wireless data communication described at FIGS. 14A-B of U.S. application Ser. No. 18/384,585 published as US20240138673A1. Implementations of an IOL in accordance with FIG. 2, including the projection system in accordance with FIG. 1, may be used with any of the embodiments of haptics described at FIGS. 18A-24 of U.S. application Ser. No. 18/373,669 published as US20250099299A1.

In various embodiments of an IOL in accordance with FIG. 2, including the projection system in accordance with FIG. 1, a distance in the Z-direction between the display 101 and the lens 103 is 700 microns to 1 mm, and a distance in the Z-direction between the lens 103 and the retina 108 is 20 mm on average for the human eye. In embodiments, the Z-direction corresponds to the optical axis (also called the pupillary axis) of the eye, which is an imaginary line perpendicular to the cornea that intersects the center of the entrance pupil, for example as illustrated in FIG. 2. In embodiments, the projection system of FIG. 1 has an overall (e.g., largest) dimension in the X-direction of 2 mm, and an overall (e.g., largest) dimension in the Y-direction of 2 mm. In embodiments, the X-direction, the Y-direction, and the Z-direction are orthogonal, for example as illustrated in FIG. 2. In embodiments, the diameter of the lens 103 in the X-direction is 1 mm and the display 101 has an overall (e.g., largest) dimension in the X-direction of 2 mm, and an overall (e.g., largest) dimension in the Y-direction of 2 mm. In this manner, the display 101 is larger than the lens 103 in the X and Y directions. In embodiments, a maximum thickness of the lens 103 in the Z-direction is 500 um. Implementations of an IOL in accordance with FIG. 2, including the projection system in accordance with FIG. 1, are not limited to these exemplary dimensions; however, implementations are sized sufficiently small to be implanted in a human eye. For example, the entire IOL is be sized to fit within a 2-3 mm incision when being implanted in the eye, wherein the fit may be achieved by folding or rolling the IOL 302, e.g., as described in U.S. application Ser. No. 18/373,669 published as US20250099299A1.

The descriptions of the various embodiments of the present disclosure have been presented for purposes of illustration, but are not intended to be exhaustive or limited to the embodiments disclosed. Many modifications and variations will be apparent to those of ordinary skill in the art without departing from the scope and spirit of the described embodiments. The terminology used herein was chosen to best explain the principles of the embodiments, the practical application or technical improvement over technologies found in the marketplace, or to enable others of ordinary skill in the art to understand the embodiments disclosed herein.

1. A device configured to be implanted in an eye, comprising:
an imaging system that receives visible light incoming to the eye; and
a projection system comprising a display and a lens that are configured to generate and project an image onto a retina of the eye in which the device is implanted, the image being based on the light received by the imaging system.

2. The device of claim 1, further comprising control circuitry that causes the display and the lens to project the image onto a determined area of the retina.

3. The device of claim 2, wherein:
the lens is between the display and the retina when the device is implanted in the eye; and
the display comprises a plurality of individually controllable light emitting elements.

4. The device of claim 2, wherein the determined area of the retina is a healthy area of the retina.

5. The device of claim 4, wherein the control circuitry determines the determined area of the retina using a stored mapping.

6. The device of claim 5, wherein the imaging system, the control circuitry, the display, and the lens are arranged in a chip stack.

7. The device of claim 6, wherein:
the imaging system is at a first side of the chip stack; and
the display and the lens are at a second side of the chip stack opposite the first side of the chip stack.

8. The device of claim 7, wherein:
the device comprises a body comprising a central portion and tabs extending outward from the central portion; and
the chip stack is in the central portion.

9. The device of claim 5, further comprising a wireless communication antenna that is configured to receive wireless communication signals from outside the device.

10. The device of claim 9, wherein the control circuitry is configured to program the mapping based on the wireless communication signals.

11. The device of claim 2, further comprising a rechargeable battery that is configured to power the imaging system, the control circuitry, and the light generation panel.

12. The device of claim 11, wherein the rechargeable battery is configured to be recharged wirelessly from a charging system located outside the eye.

13. The device of claim 1, wherein the device is configured to be implanted in a capsular bag of the eye.

14. The device of claim 1, wherein the device is configured to be implanted in a ciliary sulcus of the eye.

15. The device of claim 1, wherein the device is configured to be implanted in a chamber of the eye anterior to the iris.

16. A method comprising implanting the device of claim 1 into the eye.

17. A method of using the device of claim 1, the method comprising:
causing the device to project a diagnostic image on different locations of the retina of the eye;
receiving patient feedback for each of the different locations;
creating a mapping of the retina of the eye based on the feedback; and
programming the mapping into the device.

18. The method of claim 17, further comprising optimizing the mapping using artificial intelligence.

19. The method of claim 17, wherein the mapping maps the retina into functional areas and non-functional areas.

20. The method of claim 17, wherein the device is configured to control one or more elements of the display based on the mapping to project an image onto a functional area of the retina to reduce or eliminate a scotoma caused by a non-functional area of the retina.
